(19) United States
(12) Patent Application Publication
(10) Pub. No.: US 2007/0160636 A1
(43) Pub. Date: Jul. 12, 2007

(54) Low-density expanded cosmetic composition

(75) Inventor: Takehiko Kasai, Tokyo (JP)

Correspondence Address: OLIFF & BERRIDGE, PLC, P.O. BOX 19928, ALEXANDRIA, VA, 22320, US

(73) Assignee: L'OREAL, PARIS (FR)

(21) Appl. No.: 11/633,505

(22) Filed: Dec. 5, 2006

(30) Foreign Application Priority Data
Dec. 8, 2005 (FR) 05 53803

(51) Int. Cl. A61K 8/73

(52) U.S. Cl. 424/401; 424/70.7; 424/70.17

(57) ABSTRACT

The present invention relates to an expanded cosmetic composition for caring for and/or making up keratin materials, comprising, in a physiologically acceptable medium, at least one aqueous phase and a filler, the said composition having a density of less than or equal to 0.12 g/cm³.

The present invention relates to an expanded composition for caring for and/or making up keratin materials and to its use especially as a foundation base.

Conventionally, cosmetic compositions are usually in the form of solutions, gels, more or less fluid creams or loose or compact powders. Now, users of skincare products are increasingly in search of products that are pleasant to use and that have a novel texture.

Thus, it has already been proposed to introduce a gas, generally air, into cosmetic compositions to give them a light texture and the appearance of a mousse. This is known as expanding. The expanded emulsions obtained are appreciated for their lightness on application.

Cosmetic compositions of this type are generally in the form of temporary mousses produced just before use.

These are either aerosol products distributed from a pressurized container, with the aid of a propellent gas and thus forming a mousse, or compositions distributed from a container using a mechanical pump connected to a distribution head.

However, the expanded compositions currently available are not always entirely satisfactory, firstly in terms of quality of expanding and secondly in terms of durability of the expanding. In most cases, the mousse aspect generally generated at the time of distribution very rapidly degrades in terms of density. More specifically, the mousse very quickly has a tendency to collapse.

The present invention is directed in particular towards proposing an expanded composition of improved density, especially in terms of stability.

Thus, according to one of its aspects, the invention relates to an expanded cosmetic composition for caring for and/or making up keratin materials, comprising, in a physiologically acceptable medium, at least one aqueous phase and a filler, the said composition having a density of less than or equal to 0.12 g/cm³.

For the purposes of the invention, a physiologically acceptable medium is a medium that is compatible with the skin, the eyes and/or the hair and is thus useful for formulating a cosmetic and/or dermatological composition.

According to another of its aspects, the invention relates to a product that can form an expanded composition, comprising:
(a) a base composition comprising, in a physiologically acceptable medium, at least one aqueous phase and a filler,
(b) an expanded composition dispenser for delivering the said base composition in the form of an expanded composition with a density of less than or equal to 0.12 g/cm³, this dispenser comprising at least:
   one reservoir containing the base composition, and
   one distribution head for delivering the expanded composition.

According to another of its aspects, the invention relates to a product that can form an expanded composition as defined above, comprising:
(c) a base composition comprising, in a physiologically acceptable medium, at least one aqueous phase and a filler,
(a) an expanded composition dispenser for delivering the said base composition in the form of an expanded composition with a density of less than or equal to 0.12 g/cm³, this dispenser comprising:
   reservoir containing the base composition and a propellent gas,
   a manually activated mechanism comprising a pump to produce a volume of expanded composition, and
   distribution head, in fluid communication with the said mechanism, to deliver the said expanded composition.

According to another aspect of the invention, it concerns a process for caring for and/or making up the body, in particular the human body, especially the face, comprising the application to the surface to be treated and/or made up of an expanded composition as defined above.

For the purposes of the invention, the term “composition(s)” means the expanded cosmetic composition and/or the base composition.

The expanded composition according to the invention is advantageous in several respects. It has a light, creamy and fondant mousse texture when applied to the skin. It spreads easily on the skin and gives uniform makeup of the skin without leaving visible traces. Furthermore, after application to the skin, the makeup or the deposit obtained has a powdery, velvety finish and is comfortable to wear, without any effect of drying out or tautness; the made-up or treated skin is pleasantly soft.

Expanded Composition

The expanded compositions according to the invention are formed stably in the form of mousse using a base composition and air or an inert gas.

The air or the inert gas may represent especially from 10% to 500% and preferably from 20% to 200%, for example from 30% to 100% of the volume of the expanded composition.

This volume may be calculated by comparing the density of the base composition and of the expanded composition.

Besides air, gases that allow the expanded composition to be obtained are in particular inert gases, for example nitrogen, carbon dioxide, nitrogen oxides, noble gases or a mixture of the said gases. When the composition comprises an oxidation-sensitive compound, it is preferable to use an oxygen-free gas such as nitrogen or carbon dioxide.

The base composition serving to obtain the expanded composition has a composition similar to the expanded composition except for its higher density, insofar as it is free of air or of inert gas. The amount of gas introduced into the base composition contributes towards adjusting the density of the expanded composition to the desired value, i.e. less than or equal to 0.12 g/cm³.

As stated previously, the expanded composition of the invention has a density of less than 0.12 g/cm³. The expanded composition of the invention advantageously has a density ranging from 0.02 to 0.11 g/cm³ and preferably from 0.06 to 0.10 g/cm³, this density being measured at a temperature of about 20° C. and at atmospheric pressure according to the following protocol.

The test is performed on 25 ml of composition introduced into a 25 ml polished Plexiglas® goblet (V₁) defining a cylindrical filling space 15 mm high having a base with a diameter of 46 mm. The goblet has a bottom wall 10 mm thick and a side wall 12 mm thick.

Before measurement, the composition to be characterized and the goblet are maintained at a temperature of about 20° C. The goblet is tared and the weight value (M₁) is recorded. The expanded composition is then introduced into the goblet so as to occupy the total volume, while avoiding the formation of air bubbles during the filling of the goblet. The assembly is left to stand for 10 seconds to allow the mousse to expand fully. The top of the goblet is then skimmed before weighing (M₂). The density is assessed according to the convention ρ=(M₂−M₁)/25.

The expanded composition according to the invention shows satisfactory stability, which may be calculated by measuring the volume of mousse (V₂) remaining in the goblet after 10 minutes according to the protocol described above for the density measurement.

The ratio V₂/V₁ corresponds to the ratio between the volume of the expanded composition after 10 minutes and the volume of the expanded composition after 10 seconds.

The expression “satisfactory stability” applies especially to expanded compositions with a ratio

$$\frac{{V}_{2}}{{V}_{1}}$$

of greater than 0.85 and especially greater than 0.90, for example greater than 0.95.

For a given weight of expanded composition, the volume of the expanded composition is inversely proportional to the density of the expanded composition. Thus, the ratio between the density of the expanded composition measured after 10 seconds and the density of the expanded composition measured after 10 minutes may be greater than 0.85 and especially greater than 0.90, for example greater than 0.95.

Within the expanded composition according to the invention, the air pause may advantageously have a number-average size ranging from 20 μm to 500 μm and preferably ranging from 100 μm to 300 μm.

Aqueous Phase

The composition according to the invention generally comprises an aqueous phase comprising water and optionally a hydrophilic organic solvent, for instance monoalcohols and polyols, in particular C₂ to C₄ polyols, for example ethylene glycol, diethylene glycol, butylene glycol or glycerol.

Needless to say, the solvents are chosen as a function of their compatibility with the desired mousse texture.

The composition according to the invention may be free of glycerol so as to avoid inducing a tacky feel.

The composition advantageously comprises water. The composition may especially comprise from 35% to 95% or even from 50% to 85% water, for example from 60% to 75% water, relative to its total weight.

The composition according to the invention may also comprise seawater, especially at least 2% by weight, for example at least 5% by weight of seawater relative to the total weight.

Fillers

The composition according to the invention comprises at least one filler.

The term “fillers” should be understood as meaning colourless or white, mineral or synthetic particles of any shape, which are insoluble in the medium of the compositions irrespective of the temperature at which the composition is manufactured.

The fillers may be mineral or organic of any shape, platelet-shaped, spherical or oblong, irrespective of the crystallographic form (for example lamellar, cubic, hexagonal, orthorhombic, etc.). Mention may be made of talc, mica, silica, kaolin, polyamide (Nylon®) powders, poly-β-alanine powders, polyethylene powders, tetrafluoroethylene polymer (Teflon®) powders, lauroyllysine, starch, boron nitride, micronized titanium dioxide powders, hollow polymer microspheres such as those of polyvinylidene chloride/acrylonitrile, for instance Expancel® (Nobel Industrie), or of acrylic acid copolymers, silicone resin powders, especially silsesquioxane powders (silicone resin powders described especially in patent EP 293 795; for example Tospearls® from Toshiba), elastomeric polyorganosiloxane particles, precipitated calcium carbonate, magnesium carbonate, magnesium hydrogen carbonate, hydroxyapatite, hollow silica microspheres, glass or ceramic microcapsules, and metal soaps derived from organic carboxylic acids containing from 8 to 22 carbon atoms and preferably from 12 to 18 carbon atoms, for example zinc stearate, magnesium stearate, lithium stearate, zinc laurate or magnesium myristate; and mixtures thereof.

Advantageously, the fillers are chosen from low-density fillers. The term “low-density” means, for example, fillers with a density of less than 3.5 g/cm³ and especially less than 3 g/cm³. Without wishing to be bound to any theory, it appears that compositions comprising fillers of excessively high density form mousses of higher porosity.

The compositions may comprise low-density fillers in a content ranging from 0.5% to 30% by weight, preferably ranging from 1% to 15% by weight and preferentially ranging from 1% to 10% by weight relative to the total weight of the composition.

The compositions may also comprise fillers with a low refractive index and/or that substantially do not absorb the water and/or the oils of the composition.

A low refractive index is, for example, a refractive index of less than 1.7, for example less than 1.6. A filler that substantially does not absorb the water and/or the oils of the composition may advantageously contribute towards giving the composition a transparent appearance.

The fillers with a low refractive index and/or that substantially do not absorb the water and/or the oils of the composition may be present in the composition in a content ranging from 0.1% to 30% by weight, preferably ranging from 1% to 15% by weight and preferentially ranging from 1% to 10% by weight relative to the total weight of the composition.

Among the fillers with a refractive index of less than 1.6, which substantially do not absorb the water and the oils of the composition and which have a density of less than 3 g/cm³, mention may be made, for example, of fluorophlogopite and mixtures thereof.

Whichever they may be, the fillers may be present in the compositions in a content ranging from 0.1% to 35% by weight, preferably ranging from 0.5% to 30% by weight and preferentially ranging from 1% to 25% by weight relative to the total weight of the composition.

Advantageously, the fillers under consideration according to the invention may be surface-treated so as to improve their dispersibility within the expanded composition. Such treatments form part of the knowledge of a person skilled in the art and certain fillers thus treated are also commercially available. An example that may be mentioned is micronized (15 nm) titanium dioxide powder surface-treated with a silica/aluminium hydroxide/alginic acid mixture, sold under the name MT-100AQ.

Needless to say, the compositions according to the invention may contain fillers with a high density or refractive index or that absorb the water and/or the oil of the composition, but in an amount adjusted so as to obtain a mousse having a satisfactory density, and in particular a low porosity and satisfactory transparency.

The compositions according to the invention may thus comprise less than 3% or even less than 1.5% of barium sulfate.

Advantageously, the compositions according to the invention may comprise a polyurethane powder.

More particularly, the polyurethane powder is a powder of a copolymer of hexamethylene diisocyanate and of trimethylol hexyl lactone. Such a polyurethane powder is especially sold under the names “Plastic Powder D-400” and “Plastic Powder D-800” by the company Toshiki.

Another polyurethane powder that may be used is the product sold under the name “Plastic Powder CS-400” from the company Toshiki.

The polyurethane powder may be present in a content ranging from 0.3% to 5% by weight, preferably ranging from 0.4% to 4% by weight and preferentially ranging from 0.5% to 4% by weight relative to the total weight of the composition.

The composition of the invention may also contain at least one anionic surfactant.

This is preferably at least one foaming anionic surfactant chosen from sulfates, ether sulfates and salts thereof. Among the salts of sulfates and of ether sulfates, the sodium and triethanolamine salts are preferentially chosen. The foaming anionic surfactant that may thus be used is sodium lauryl ether sulfate, and especially the product sold under the name Texapon by the company Henkel.

In general, the composition of the invention contains an amount of anionic surfactant ranging from 0.5% to 50% by weight and preferably from 2% to 30% by weight relative to the total weight of the composition.

The composition according to the invention may also contain an emulsifier that may be chosen from any emulsifier conventionally used for O/W emulsions.

Examples of emulsifiers that may be mentioned include:

(1) nonionic surfactants with an HLB of greater than or equal to 9, such as oxyethylenated fatty acid esters of glycerol; oxyethylenated fatty acid esters of sorbitan; oxyethylenated fatty acid derivatives; fatty acid esters of a sugar and especially fatty esters of sucrose such as sucrose stearate, for instance the product sold under the name Tegosoft PSE 141G by the company Goldschmidt; alkyl polyglucoside ethers, and mixtures thereof;

(2) silicone emulsifiers such as oxyethylenated polydimethylsiloxanes (dimethicone copolyols), for instance the product sold under the name “DC2-5695” by the company Dow Corning.

The composition according to the invention may comprise, for example, from 0.5% to 50%, preferably from 2% to 15% and better still from 4% to 10% by weight of emulsifier(s) relative to the total weight of the composition.

The composition according to the invention generally comprises a solid or liquid fatty substance and especially a liquid fatty substance.

The composition according to the invention may thus comprise an oily phase comprising at least one oil that may be chosen from volatile oils and non-volatile oils, and mixtures thereof. Advantageously, the composition comprises at least one volatile oil and at least one non-volatile oil.

The composition according to the invention may comprise at least one volatile oil.

For the purposes of the invention, the term “volatile oil” means any oil that is capable of evaporating on contact with the skin, at room temperature and atmospheric pressure. The volatile oils of the invention are volatile cosmetic oils that are liquid at room temperature, with a non-zero vapour pressure at room temperature and atmospheric pressure, ranging in particular from 0.13 Pa to 40,000 Pa (0.001 to 300 mmHg) and preferably ranging from 1.3 Pa to 1300 Pa (0.01 to 10 mmHg).

The volatile oil may be chosen from volatile hydrocarbon-based oils, volatile silicone oils and volatile fluoro oils, and mixtures thereof.

The term “hydrocarbon-based oil” means an oil mainly containing hydrogen and carbon atoms and possibly oxygen, nitrogen, sulfur and/or phosphorus atoms.

The volatile hydrocarbon-based oils may be chosen from hydrocarbon-based oils containing from 8 to 16 carbon atoms, and especially branched C₈-C₁₆ alkanes, for instance C₈-C₁₆ isoalkanes of petroleum origin (also known as isoparaffins), for instance isododecane (also known as 2,2,4,4,6-pentamethylheptane), isodecane and isohexadecane, for example the oils sold under the trade names Isopar® and Permethyl®.

Volatile oils that may also be used include volatile silicones, for instance volatile linear or cyclic silicone oils, especially those with a viscosity ≦5 centistokes (5×10-6 m²/s) and especially containing from 2 to 10 silicon atoms and preferably from 2 to 7 silicon atoms, these silicones optionally comprising alkyl or alkoxy groups containing from 1 to 10 carbon atoms. As volatile silicone oils that may be used in the invention, mention may be made especially of octamethyl cyclotetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane and dodecamethyl pentasiloxane, and mixtures thereof.

Volatile oils that may also be used are volatile fluoro oils: mention may be made of nonafluoroethoxybutane, nonafluoromethoxybutane, decafluoropentane, tetradecafluorohexane and dodecafluoropentane, and mixtures thereof.

The composition according to the invention may comprise at least one non-volatile oil.

The term “non-volatile oil” means an oil that remains on the skin at room temperature and atmospheric pressure for at least several hours and that especially has a vapour pressure of less than 0.13 Pa (0.01 mmHg).

These non-volatile oils may be hydrocarbon-based oils especially of animal or plant origin, or silicone oils, or mixtures thereof. The term “hydrocarbon-based oil” means an oil mainly containing hydrogen and carbon atoms and possibly oxygen, nitrogen, sulfur and/or phosphorus atoms.

The non-volatile oils may be chosen especially from non-volatile hydrocarbon-based oils, which may be fluorinated, and/or non-volatile silicone oils.

Non-volatile hydrocarbon-based oils that may especially be mentioned include:
hydrocarbon-based oils of animal origin,
hydrocarbon-based oils of plant origin, such as triglycerides consisting of fatty acid esters of glycerol, the fatty acids of which may have varied chain lengths from C₄ to C₂₄, these chains possibly being linear or branched, and saturated or unsaturated; these oils are especially heptanoic or octanoic acid triglycerides, or alternatively wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, sesame seed oil, marrow oil, rapeseed oil, blackcurrant oil, evening primrose oil, millet oil, barley oil, quinoa oil, rye oil, safflower oil, candlenut oil, passionflower oil or musk rose oil; shea butter; or caprylic/capric acid triglycerides, for instance those sold by the company Stéarineries Dubois or those sold under the names Miglyol 810®, 812® and 818® by the company Dynamit Nobel,
synthetic ethers containing from 10 to 40 carbon atoms,
linear or branched hydrocarbons of mineral or synthetic origin, such as petroleum jelly, polydecenes, hydrogenated polyisobutene such as Parleam®, squalane and liquid paraffins, and mixtures thereof,
synthetic esters, for instance oils of formula R₁COOR₂ in which R₁ represents a linear or branched fatty acid residue containing from 1 to 40 carbon atoms and R₂ represents a hydrocarbon-based chain, which is especially branched, containing from 1 to 40 carbon atoms, on condition that R₁+R₂≧10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, 2-hexyldecyl laurate, 2-octyldecyl palmitate, 2-octyldodecyl myristate, or alcohol or polyalcohol heptanoates, octanoates, decanoates or ricinoleates, for instance propylene glycol dioctanoate; hydroxylated esters, for instance isostearyl lactate, diisostearyl malate or 2-octyldodecyl lactate; polyol esters and pentaerythritol esters,
fatty alcohols that are liquid at room temperature with a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol,
higher fatty acids such as oleic acid, linoleic acid or linolenic acid, and mixtures thereof.

The non-volatile silicone oils that may be used in the composition according to the invention may be non-volatile polydimethylsiloxanes (PDMS), polydimethylsiloxanes comprising alkyl or alkoxy groups, which are pendent and/or at the end of a silicone chain, these groups each contain from 2 to 24 carbon atoms, phenyl silicones, for instance phenyl trimethicones, phenyl dimethicones, phenyltrimethylsiloxydiphenylsiloxanes, diphenyl dimethicones and diphenylmethyldiphenyltrisiloxanes, and mixtures thereof.

The composition according to the invention advantageously comprises from 1% to 40% by weight, preferably from 2% to 30% by weight and better still from 5% to 20% by weight of oily phase relative to its total weight.

The other fatty substances that may be present in the oily phase may be, for example, fatty acids, fatty alcohols such as cetyl alcohol, and waxes.

Thus, the fatty phase may also comprise compounds that are solid at room temperature (20° C.) such as waxes, these components possibly improving the stability of the expanded composition. These compounds are added either in molten form or in solid form to the oily phase, heated to a temperature above the melting point of the solid. These compounds may be waxes or compounds similar to waxes, for example natural renewable waxes (insect waxes, animal waxes and plant waxes), fossil waxes (petroleum wax, lignite wax, peat wax or ozokerite), synthetic waxes (Fischer-Tropsch waxes, polyethylene waxes or amide waxes), high-melting paraffins, esters, fats, long-chain carboxylic acids or long-chain alcohols of C₁₀ to C₂₂, each of these waxes having a melting or solidification point of above room temperature (20° C.).

As non-limiting illustrations of base compositions that are suitable for the invention, mention may be made more particularly of those combining as surfactants a cetyl alcohol, a cetearyl alcohol and/or PEG-10-dimethicone and at least one water-insoluble pulverulent compound such as a polyurethane powder, for instance Plastic Powder D-800 or CS-400® or surface-treated TiO₂, for instance the commercial product MT-100AQ.

The composition according to the invention may comprise at least one dyestuff chosen especially from pigments, nacres, liposoluble dyes and water-soluble dyes, and mixtures thereof.

The term “pigments” should be understood as meaning white or coloured, mineral or organic particles of any shape, which are insoluble in the physiological medium and are intended to colour the composition.

The term “nacres” should be understood as meaning iridescent particles of any shape, especially produced by certain molluscs in their shell or else synthesized.

The term “dyes” should be understood as meaning generally organic compounds that are soluble in water or in fatty substances such as oils.

The pigments may be white or coloured, and mineral and/or organic. Among the mineral pigments that may be mentioned are titanium dioxide, optionally surface-treated, zirconium oxide and cerium oxide, and also zinc oxide, iron oxide (black, yellow or red) or chromium oxide, manganese violet, ultramarine blue, chromium hydrate and ferric blue, and metal powders such as aluminium powder or copper powder.

Among the organic pigments that may be mentioned are carbon black, pigments of D&C type and lakes based on cochineal carmine or on barium, strontium, calcium or aluminium.

The nacreous pigments may be chosen from white nacreous pigments such as mica coated with titanium or with bismuth oxychloride, coloured nacreous pigments such as titanium mica coated with iron oxides, titanium mica coated especially with ferric blue or with chromium oxide, titanium mica coated with an organic pigment of the abovementioned type, and also nacreous pigments based on bismuth oxychloride.

The liposoluble dyes are, for example, Sudan red, D&C Red No 17, D&C Green No 6, β-carotene, soybean oil, Sudan brown, D&C Yellow No 11, D&C Violet No 2, D&C Orange No 5, quinoline yellow, annatto and bromo acids.

The dyestuffs may be present in a content ranging from 0.1% to 30% by weight, preferably ranging from 0.1% to 20% by weight, preferably ranging from 0.5% to 15% by weight, preferentially ranging from 1% to 15% by weight and more preferentially ranging from 5% to 15% by weight relative to the total weight of the composition.

In a known manner, the composition of the invention may also contain adjuvants that are common in cosmetics, such as humectants, preserving agents, antioxidants, complexing agents, solvents, fragrances, screening agents, bactericides, odour absorbers, sunscreens, vitamins, moisturizers, self-tanning compounds and antiwrinkle active agents. The amounts of these various adjuvants are those conventionally used in the field under consideration, for example from 0.01% to 20% of the total weight of the composition. Depending on their nature, these adjuvants may be introduced into the fatty phase, into the aqueous phase and/or into lipid vesicles.

The expanded composition according to the invention finds its application in a wide variety of treatments, especially cosmetic treatments, of the skin, the lips and the hair, including the scalp, especially for treating, protecting or caring for the skin, the lips and/or the hair, and/or for making up the skin and/or the lips. It may also be intended for treating dry skin and/or dry lips.

Thus, a subject of the invention is also the cosmetic use of the expanded composition as defined above for treating, protecting or caring for the skin, the lips and/or the hair, and/or for making up the skin and/or the lips, in particular for preparing a foundation or a foundation base.

A subject of the invention is also the use of a product as defined above to form an expanded composition serving as a base for the preparation of a makeup of foundation type.

Needless to say, a person skilled in the art will take care to select this or of these optional additional compound(s), and/or the amount thereof, such that the advantageous properties of the composition according to the invention are not, or are not substantially, adversely affected by the envisaged addition.

The expanded composition may be obtained from a base composition, i.e. a composition formed from the same ingredients as those present in the expanded composition, but free of air or gas.

For example, the base composition may be placed in a beater and beaten with gas. The mousse then forms, and its density can be adjusted. The expanded composition in mousse form can then be stored in a container.

Alternatively or in addition, the expanded composition may be obtained from a base composition in an expanded composition distributor. This distributor may be an aerosol containing, besides the base composition, a propellent gas.

This propellent gas may represent less than 20% of the base composition and in particular may represent from 1% to 10% of the total weight of the base composition. The propellent gas that may be used may be chosen from carbon dioxide, nitrogen, nitrous oxide and volatile hydrocarbons such as butane, isobutane, propane, ethane, pentane, isododecane or isohexadecane, and mixtures thereof.

The base composition may also not contain any propellent gas. In this case, the base composition may be in an expanded composition distributor comprising a distribution head for delivering the expanded composition, a pump and a dip tube for transferring the composition from the container into the head to deliver the product. The expanded composition is formed by forcing the base composition to pass through a material comprising a porous substance such as a sintered material, a plastic or metallic screening grate, or similar structures.

Such dispensers are well known to those skilled in the art and are described in patents U.S. Pat. No. 3,709,437 (Wright), U.S. Pat. No. 3,937,364 (Wright), U.S. Pat. No. 4,022,351 (Wright), U.S. Pat. No. 4,147,306 (Bennett), U.S. Pat. No. 4,184,615 (Wright), U.S. Pat. No. 4,598,862 (Rice), U.S. Pat. No. 4,615,467 (Grogan et al.) and U.S. Pat. No. 5,364,031 (Tamiguchi et al.).

The example below is given as a non-limiting illustration.

### EXAMPLE 1

The base composition below is a foundation base. The indices relating to each of the compounds specify their respective commercial name.

| | | Concentration |
|---|---|---|
| Phase | INCI Name | (mass %) |
| A | Crosslinked polymer of HDI/trimethylol | 0.950 |
| | hexyl lactone (and) silica ¹ | |
| | Calcium carbonate ² | 3.012 |
| | Titanium dioxide (and) silica (and) aluminium | 2.375 |
| | hydroxide (and) alginic acid ³ | |
| | Synthetic fluorophlogopite (and) | 7.534 |
| | hydrogenated egg oil (and) methicone ⁴ | |
| | Iron oxides (and) C₉-C₁₅ fluoroalkyl | 0.133 |
| | phosphate ⁵ | |
| | Iron oxides (and) C₉-C₁₅ fluoroalkyl | 0.038 |
| | phosphate ⁶ | |
| | Iron oxides (and) C₉-C₁₅ fluoroalkyl | 0.029 |
| | phosphate ⁷ | |
| B | Steareth-10 ⁸ | 0.399 |
| | Chlorphenesine | 0.238 |
| | Methylparaben | 0.285 |
| | Cetearyl alcohol (and) polysorbate 60 ⁹ | 0.665 |
| | Ethylhexyl methoxycinnamate ¹⁰ | 4.940 |
| | Cetyl alcohol ¹¹ | 0.143 |
| | Caprylyl glycol ¹² | 0.285 |
| C | Water | 64.872 |
| | Seawater | 5.700 |
| | Dipotassium glycyrrhizate | 0.190 |
| D | Propane | 1.160 |
| | Butane | 2.670 |
| | Isobutane | 1.120 |
| | Ethane | 0.005 |
| | Pentane | 0.045 |
| E | PEG-10 dimethicone ¹³ | 0.694 |
| | Butylene glycol ¹⁴ | 1.900 |
| | Phenoxyethanol | 0.238 |
| | Tocopheryl acetate ¹⁵ | 0.048 |
| | Polysorbate 20 | 0.209 |
| | Fragrance ¹⁶ | 0.019 |
| | Propylene glycol (and) hexylene glycol | 0.095 |
| | (and) extract of Hamamelis virginiana ¹⁷ | |

¹ Plastic Powder D 400
² Socal 90 A
³ MT-100 A Q
⁴ SNI - Synthetic Mica PDM - 8 W
⁵ PF5 Yellow 601
⁶ PF5 Red R516L
⁷ PF5 Black BL100
⁸ Brij 76
⁹ Polawax NF
¹⁰ Uvinul MC 80
¹¹ Lanette 16
¹² Dermosoft Octiol
¹³ SH 3746 Oil
¹⁴ 1,3-Butylene glycol
¹⁵ DL-α-Tocopheryl acetate
¹⁶ LIPS - 1086C
¹⁷Hamamelis essence on support 36 HMW 1090

With a suitable device, it is possible to form, using this base composition, an expanded composition with a density of 0.0868 g/cm³ that shows satisfactory stability.

1. Expanded cosmetic composition for caring for and/or making up keratin materials, comprising, in a physiologically acceptable medium, at least one aqueous phase and a filler, the composition having a density of less than or equal to 0.12 g/cm³.

2. Composition according to claim 1, having a density ranging from 0.02 to 0.11 g/cm³.

3. Composition according to claim 2, having a density ranging from 0.06 to 0.10 g/cm³.

4. Composition according to claim 1, for application to the skin.

5. Composition according to claim 4, for application to the human facial skin.

6. Composition according to claim 5, in the form of a foundation or a foundation base.

7. Composition according to claim 1, comprising from 35% to 95% by weight of water relative to its total weight.

8. Composition according to claim 1, comprising at least one surfactant.

9. Composition according to claim 8, comprising 2% to 50% by weight of surfactant(s) relative to its total weight.

10. Composition according to claim 8, wherein the surfactant is chosen from alkylpolyglucosides, maltose esters, polyglycerolated fatty alcohols, glucamine derivatives, carboxylates, amino acid derivatives, alkyl sulfates, alkyl ether sulfates, sulfonates, isethionates, taurates, sulfosuccinates, alkyl sulfoacetates, phosphates and alkyl phosphates, polypeptides, anionic derivatives of alkylpolyglucosides, fatty acid soaps, betaines, N-alkylamido betaines and derivatives thereof, glycine derivatives, sultaines, alkyl polyaminocarboxylates and alkylamphoacetates, and mixtures thereof.

11. Composition according to claim 8, wherein the surfactant is a foaming surfactant.

12. Composition according to claim 1, also comprising at least one solvent chosen from monoalcohols and polyols, and mixtures thereof.

13. Composition according to claim 1, comprising from 0.1% to 35% by weight of filler(s) relative to its total weight.

14. Composition according to claim 1, wherein the filler is chosen from talc, mica, silica, kaolin, polyamide powders, poly-β-alanine powders, polyethylene powders, tetrafluoroethylene polymer powders, lauroyllysine, starch, boron nitride, micronized titanium dioxide powders, hollow polymer microspheres such as those of polyvinylidene chloride/acrylonitrile or of acrylic acid copolymers, silicone resin powders, especially silsesquioxane powders, elastomeric polyorganosiloxane particles, precipitated calcium carbonate, magnesium carbonate, magnesium hydrogen carbonate, hydroxyapatite, hollow silica microspheres, glass or ceramic microcapsules, and metal soaps derived from organic carboxylic acids containing from 8 to 22 carbon atoms, and mixtures thereof.

15. Composition according to claim 1, comprising at least one filler with a density of less than 3.0 g/cm³.

16. Composition according to claim 1, comprising at least one filler with a refractive index of less than 1.60.

17. Composition according to claim 1, comprising as filler at least fluorophlogopite.

18. Composition according to claim 1, comprising at least one polyurethane powder.

19. Composition according to claim 1, comprising a volatile hydrocarbon-based oil.

20. Product that can form an expanded composition, comprising:
(a) a base composition comprising, in a physiologically acceptable medium, at least one aqueous phase and a filler,
(b) an expanded composition dispenser for delivering the base composition in the form of an expanded composition with a density of less than or equal to 0.12 g/cm³, this dispenser comprising at least:
one reservoir containing the base composition, and
one distribution head for delivering the expanded composition.

21. Product that can form an expanded composition, comprising:
(a) a base composition comprising, in a physiologically acceptable medium, at least one aqueous phase and a filler,
(b) an expanded composition dispenser for delivering the base composition in the form of an expanded composition with a density of less than or equal to 0.12 g/cm³, this dispenser comprising:
a reservoir containing the base composition and a propellent gas,
a manually activated mechanism comprising a pump to produce a volume of expanded composition, and
a distribution head, in fluid communication with the mechanism, to deliver the said expanded composition.

22. Product according to claim 21, wherein the propellent gas is chosen from propane, butane, isobutane, ethane, pentane, isododecane, isodecane and isohexadecane, and mixtures thereof.

23. Product according to claim 20, wherein the expanded composition is for caring for and/or making up keratin materials, comprising, in a physiologically acceptable medium, at least one aqueous phase and a filler, the composition having a density of less than or equal to 0.12 g/cm³ , the composition having a density ranging from 0.06 to 0.10 g/cm³.

24. Process for caring for and/or making up the body, comprising:
the application to the surface to be treated and/or made up of an expanded composition as defined in claim 1.
